(19) United States
(12) Patent Application Publication
(10) Pub. No.: US 2012/0175273 A1
(43) Pub. Date: Jul. 12, 2012

(54) Toothpaste tablet

(76) Inventors: Scott Jacobs, Lakewood, CO (US); Allison J. LeGendre, Bainbridge Island, WA (US); David Wood, Omaha, NE (US)

(21) Appl. No.: 12/930,475

(22) Filed: Jan. 7, 2011

(51) Int. Cl. A61J 1/03; B29C 43/02; A61Q 11/00; A61K 8/97; A61K 8/21

(52) U.S. Cl. 206/63.5; 424/49; 424/52; 424/58; 264/330

(57) ABSTRACT

A toothpaste tablet having a tablet binder in an amount sufficient to form a tablet, a surfactant in amount sufficient to reduce surface tension and increase the rate of dissolution of the tablet in contact with saliva, a cranberry extract in an amount sufficient to reduce micro-adhesion of bacteria, an abrasive in an amount sufficient to mechanically remove debris from a tooth surface and debride plaque, and an artificial sweetener in an amount sufficient to adjust the flavor of the tablet when chewed by a user.

### FIELD OF THE INVENTION

The present invention relates to a toothpaste tablet used for cleaning one's teeth. More particularly, the invention relates to a toothpaste tablet that is stable until used and that dissolves quickly in the mouth prior to brushing.

### BACKGROUND OF THE INVENTION

Virtually all dentifrices found in the current marketplace are in paste format and packaged in a tube, squeeze bottle, pressurized can, or pump dispenser. These have found good market acceptance even though the devices requires the user to hold the toothbrush in one hand and the dispenser in the other hand. Also, traditional toothpastes rely on the combination of hydrated glycol/glycerin/surfactant content to create a foaming action in the mouth when brushing.

Alternative toothpaste forms have been considered. One is an effervescent tablet that must be kept in a hermetically sealed package because moisture causes the effervescence to begin regardless if it is desired.

It would be of advantage in the art if an alternative source of toothpaste could be provided that is safe, simple to use, and more effective than conventional toothpastes.

Other advantages will appear hereinafter.

### SUMMARY OF THE INVENTION

It has now been discovered that the above and other advantages of the present invention may be obtained in the following manner. Specifically, the present invention provides a unit dose toothpaste tablet that is chemically stable and sanitary, compared to conventional toothpastes.

The tablet is an enhanced monomeric water soluble vehicle that directly affects the properties of tooth surface affinity and bacterial inhibition. When inserted into the mouth, chewed and brushed to activate foaming, there is an instantaneous spike in pH, a subsequent buffering upon dissolution in the mouth, followed by enhanced saturation of teeth and oral mucosa, and a total absence of disturbance or harm to the hydroxyapatite crystalline matrix on the surface of the teeth. The effect of chewing force on the tablet facilitates penetration of hard-to-reach interproximal tooth surfaces using tensile and compressive force.

### DESCRIPTION OF DRAWINGS

FIG. 1 is a perspective view of a device for dispensing the tablets according to the present invention.

FIG. 2 is a perspective view of the device of FIG. 1, shown in the closed or shut position.

### DETAILED DESCRIPTION OF THE PREFERRED EMBODIMENT

The present invention provides for substantial improvements in a toothpaste tablet. The tablet is a unit dose tablet ranging in size from 200 mg-5000 mg (optimum size ranging from 250 mg-1000 mg), most commonly manufactured as a 500 mg or 1 g tablet. The advantages of this dry tablet over the traditional wet dentifrice are to include increased chemical stability and unique sanitary packaging. Due to the dry state of the toothpaste tablet verses the aqueous state of traditional toothpaste, ingredients remain stable prior to contact with the oral environment, thus resulting in enhanced mouth activity in addition to a more sustainable shelf life.

Specifically, the toothpaste tablet includes a tablet binder in an amount sufficient to form a tablet, a surfactant in amount sufficient to reduce surface tension and increase the rate of dissolution of the tablet in contact with saliva along with assisting in foaming the tablet, an anthiocyanad(s) extract such as cranberry extract in an amount sufficient to reduce micro-adhesion of bacteria and reduce the levels of bacteria both in the oral cavity and on the toothbrush, an abrasive in an amount sufficient to mechanically remove debris from a tooth surface and debride plaque, and an artificial sweetener in an amount sufficient to adjust the flavor of the tablet when chewed by a user.

The tablet of this invention may also include a food dye to provide a specific color to mask the color of other additives and assists in adjusting the color of the tablet when it has been activated in the mouth. When dissolved in the mouth, the toothpaste tablet of this invention may contain a buffer in an amount sufficient to adjust the pH when dissolved in the mouth from about 6.0 to about 8.0. In addition, the cranberry extract provides an initial decrease in pH to below 6.5 and the buffering agent in an amount sufficient to restore the pH to the range specified.

The natural and artificial sweeteners are selected from at least one of the group consisting of polysaccharides, artificial polyols, natural polyols coming from fruits and trees, highly refined fruits (example Lou Han Gue), purified herbs, green tea, mongloicides, white tea, stevia, carrageenan, cranberry, natural pectin, alginic acid, xanthan gum, hyaluronic acid, chondroitin sulfate, gum Arabic, gum karaya, gum tradgacanth, chitosan, starch, dextrins, Neotame, Sucralose, Acefulame K, Saccharin(s), Cyclomates, Apartame, Licorice root, Glycyrrhizin extract, Citrguar gum, guar gum, lactose, glyhrician manufactured under the brand name Magnasweet and mixtures thereof. The amount of natural or artificial sweeteners range from about 0.05% to about 10% by weight, based on the weight of the tablet. Also, if sorbital or other binders are used, they may also function as sweeteners so that the amount of sweetener will be much more. The percent of sweeteners will range from about 10% about 95% by weight.

The binder ranges from about 10% to about 90% by weight, based on the weight of the tablet. The binders preferably are polyols, such as mannitol, Maltitol, sorbitol, isomalt, erythritol and xylitol or invert sugars. Other binders include calcium phosphate, calcium carbonate, sodium phosphate, sodium bicarbonate, sodium carbonate, sodium phosphate, potassium phosphate, potassium carbonate, polyethylene glycol and esters thereof. A preferred binder(s) is sorbitol, xylitol. They function as a necessary binder but also provide sweetness.

The amount of surfactant ranges from about 1.0% to about 5% by weight, based on the weight of the tablet.

The amount of cranberry extract ranges from about 0.1% to about 10% by weight of the pure extract, based on the weight of the tablet. If diluted cranberry extracts are used, the weight percent will vary. The cranberry extract used in the tablet has an active natural ingredient classified as anthocyanins, polyphenolics and catechins which helps to reduce the formation of plaque as well as reduce harmful levels of oral bacteria and virus. Other extracts from dark berries such as blueberries, blackberries, choke berries, cherries, raspberries and purple grapes also contain anthocyanins, polyphenolics and catechins and can be used in place of the cranberry extract. In addition, certain spices such as nutmeg contain anthocyanins, polyphenolics and catechins and can be used in place of the cranberry extract.

The amount of abrasive ranges from about 0.1% to about 20% by weight, based on the weight of the tablet. The abrasive is selected from dehydrated silica, hydrated silica, calcium carbonate and surface treated carbonate salts or phosphates, and mixtures thereof.

The surfactant utilized in the toothpaste tablet is uniquely efficacious, as a result of the creation of an initial thin, serous substrate (as opposed to dextrin formation) as the dry surfactant mixes with saliva. This thin substrate has the ability to optimally saturate teeth and oral mucosa. In contrast, traditional dentifrices create a thicker, less penetrable substrate. The surfactant activity found in the toothpaste tablet creates a synergistic effect, as the thinner substrate allows the freshly hydrated ingredients optimum coverage in addition to the optimum efficacy. Both simple and combined surfactant activities are unique to the toothpaste tablet. In the preferred embodiment, sodium lauryl sulfate (SLS, in a concentration level of 0.005% to 20%, and preferably in a range of 1% to 8% with the preferred embodiment at 2%-3% by weight) is utilized. Sodium dodecyl sulfate, SDS, as well as sodium lauroly sarcosine, stearoly sarconsine, sucrose cocoate, potassium cocoate, sodium lauryl sarcosonate, sodium glutimate, lauryl glutimate, potassium lauryl glutimate marketed under the trade name Texapon K12, glucose oxydase, lactoperoxidase and the like may be employed to enhance surfactant activity.

Chemically, the unique role of cranberry extract as noted in European Patent No. WO2009141524, the disclosure of which is incorporated herein by reference in its entirety, delivered as a unique enhanced monomeric water soluble vehicle, dramatically affects the properties of tooth surface affinity and bacterial inhibition. Several synergistic phenomena occur, and include 1.) an instantaneous spike in pH, 2.) subsequent buffering upon dissolution in the mouth, 3.) enhanced saturation of teeth and oral mucosa, and 4.) an absence of disturbance or harm to the hydroxyapatite crystalline matrix on the surface of the teeth. Mechanically, the effect of occlusal (chewing) force on the tablet uniquely facilitates penetration of hard-to-reach interproximal tooth surfaces through tensile and compressive force. Other anthiocyanad extracts may also function in this manner.

It is also contemplated that the tablet of this invention may be made from all natural/organic ingredients. The version of the tablet with a formulation containing all natural/organic ingredients includes natural/organic sources of the original formulation, and may include polysaccharides from purified herbs, green tea, white tea, carrageenan, cranberry, natural pectin and inulin. Specific anionic polysaccharides, and nonionic polysaccharides are cited below. Organic/natural surfactant sources may include yucca, slippery elm bark, soap bark extract, arginine cocoate or n-acetyl gutamine cocoate, sodium n-cocoyl-glutamate, or forms of lauro stearoly. Additional proanthocyanidins, such as grapeseed extract, are also cited.

Delivery simply involves placing one tablet in the mouth, immediately chewing the tablet, and upon experiencing tablet dissolution, brushing with regular or electric tooth brush to generate foaming and cleaning, and then expectorating. The user may also elect to swallow the dissolved tablet after brushing. While it is contemplated that the toothpaste tablet of this invention is to be used with a manual or electric tooth brush, it is also contemplated that the user may elect to chew the tablet, swish the dissolved tablet in the oral cavity and either swallow or rinse after using. By not using a manual or electric tooth brush, the ingredients in the tablet of this invention have a positive effect on overall oral health.

The toothpaste tablet uses a dry form of mild abrasive which also acts as a desiccant until tablet comes in contact with saliva. In the preferred embodiment, dehydrated or granulated hydrated silica, in the amount recommended for safe daily tooth abrasion, is utilized. This diverges from the use of hydrated silica, saturated in an aqueous environment at the time of manufacture, found in traditional dentifrices. The dry state of silica found in the toothpaste tablet assists in keeping package moisture levels to a minimum, resulting in increased chemical stability without preservatives. In the natural/organic version of the tablet, ground shells from nuts or fruit stones, or modified cellulose, may be used as gentle abrasives with desiccant properties.

It is also contemplated that the toothpaste tablet of this invention may include a quantity of fluoride. Fluoride is a controlled substance by the FDA, and because the tablet of this invention is a unit dose tablet, appropriate control is achieved. Fluoride will also be more stable in the dry tablet than in a wet paste. Examples of fluorides are sodium fluoride, sodium monofluorophosphate, calcium fluoride, stannous fluoride, silver diamine fluoride and slow release fluoride, which are any of the fluorides that have been coated with a polyol. The amount of fluoride may range from about 0.01% to about 5.0% by weight. And preferably about 1.0% by weight.

The preferred embodiment of the toothpaste tablet extends the flavor over time. The specific formulation layers natural flavors including, but not limited to, the cranberry extract, menthol, and peppermint and spearmint extracts and additional flavors. This layering of flavors is preserved in a non-aqueous and more stable environment until the tablet is saturated with saliva, at which time the agglomeration releases these flavor extracts.

In the manufacturing process of the toothpaste tablet, the run parameters are specifically adjusted for this unique formulation, such that excess heat is not required to form a tablet. The result is a tablet with sufficient hardness and dissolvability. Specifically, the speed and temperature settings are cooler than usual tablet runs. This keeps active ingredients from breaking down during the manufacturing process. Temperatures at the time of manufacturing including batching, measuring, and pressing tablets do not exceed 78 degrees F. (versus a typical manufacturing temperature of >90 degrees F.). The temperature regulation is most critical in process at the point of formulation within the turret, optimizing tablet agglomeration and proanthocyanidin bioactivity from cranberry extracts and other fruit extracts. This process protects proteins from denaturation, which allows boactives to remain active.

The tablets of this invention may be any convenient size, and should be large enough to produce an adequate amount of foamed liquid for brushing or otherwise using the tablet in the oral cavity. Round 500 mg tablets have been formulated having a diameter of 0.439 inches (11.82 mm). Other geometric shapes such as a capsule format, and bi-layered tablets and/or pre-molded slugs in a tablet or tablet in a tablet are also contemplated here.

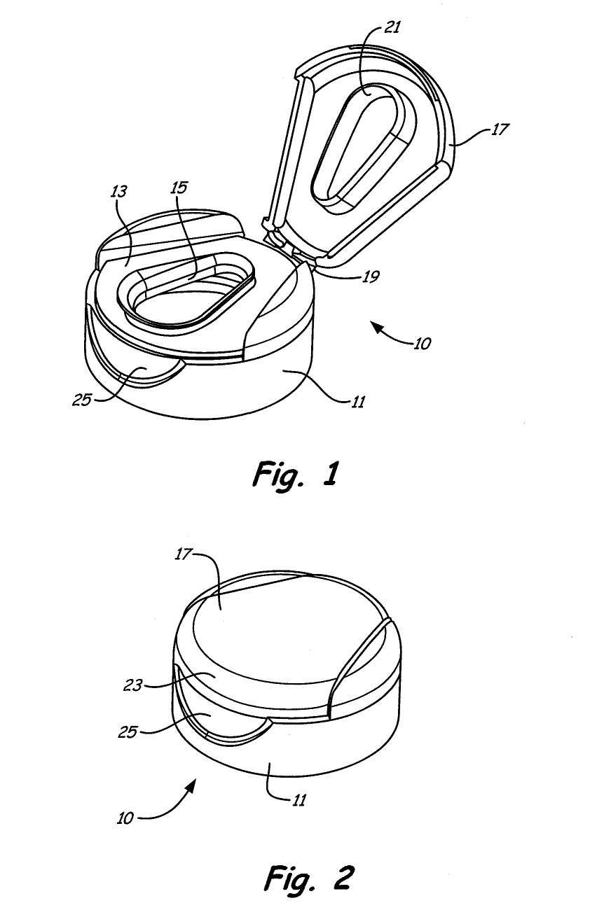

FIG. 1 illustrates a device for dispensing the tablets of this invention. Dispenser 10 generally includes a base portion 11 for holding a plurality of tablets, such as a supply for a week or a month. Covering base 11 is inner lid or layer 13 having an opening 15 through which a tablet is dispensed. Inner lid 13 may be attached by snaps or by a hinge, not shown, to permit the manufacturer to put tablets inside base 11 and have a closed attachment between base 11 and layer 13.

Lid 17 is attached to base 11 by hinge 19. Lid 17 also has a seal member 21 on the inside thereof that is sized to fill and seal opening 15 to preserve the tablets remaining in base 11. When lid 17 closes on base 11 as in FIG. 2, the end 23 of lid 17 extends over recess 25 to facilitate opening 17 with one hand when a new tablet is desired.

Opening 15 is shown as elliptical and at its widest part is about 1% to about 10% smaller, and preferably 3% smaller than the diameter of the tablet, which in the above example, would be about 0.426 inches (10.82 mm). The ratio of the length to width of elliptical opening 15 is about 2 to 1 to allow for controlled distribution of one tablet each time dispenser 10 is shaken. If desired, a maximum of two tablets can be shaken out for use. Because the possibility of dispensing more than a maximum of two tablets, dispenser 10 controls the possibility of contamination in a bathroom environment. It also prevents waste of tablets. Dispenser 10 also controls the level of humidity in the tablets. Most preferred is a width of opening 15 that is from about 1% to about 10% smaller than the width of the tablet. If capsules are used instead of round tablets, opening 15 should not exceed ⅔ of the longest dimension of the capsule to insure that only one exit upon shaking.

The method of dispensing the tablet is unique, using a flip-top cap, which reduces the tendency for more than one tablet to be extracted from the package at a time. Specifically, as described above, a modified elliptical shaped orifice with a re-closable cap, which can easily be operated by one hand, is tipped thus allowing one tablet to dispense at a time. The style of cap is unique to tablets and was originally designed to dispense liquid in increments. This flip-top dispenser, with modified elliptical opening of less than 85% of the bottle's diameter, is totally unique and applicable for unit dose tablets in any geometric configuration. This unique dispensing format also makes the tablet more sanitary, since contact with bacterial and viral infected surfaces is minimized prior to tablet placement in the mouth. This is in stark contrast to the unsanitary conditions found in a paste dispensing method of a tube, squeeze bottle, or pressurized can which can carry microbes after being swiped along the surface of the dispensing orifice with a used toothbrush containing microbes from the mouth and ambient airborne microbes. The dry tablet format also has the unique ability to be dispensed from a bulk bin, allowing the consumer to determine individual weight and volume.

While particular embodiments of the present invention have been illustrated and described, it is not intended to limit the invention, except as defined by the following claims.

1. A toothpaste tablet for use in brushing the user's teeth, comprising:
a tablet binder in an amount sufficient to form a tablet, a surfactant in amount sufficient to reduce surface tension and increase the rate of dissolution of the tablet in contact with saliva, a anthiocyanin extract in an amount sufficient to reduce micro-adhesion of bacteria, an abrasive in an amount sufficient to mechanically remove debris from a tooth surface and debride plaque, and a sweetener in an amount sufficient to adjust the flavor of the tablet when chewed by a user.

2. The tablet of claim 1, which further includes a dental fluoride in an amount ranging from 0.01% to 5% by weight.

3. The tablet of claim 1, which further contains a buffer in an amount sufficient to adjust the pH when dissolved in the mouth to from about 6.0 to about 8.0.

4. The tablet of claim 3, wherein the anthiocyanin extract is selected from the group consisting of a cranberry anthocyanin polyphenolic and catechin extract that provides an initial decrease in pH to below 6.5 and the buffering agent in an amount sufficient to restore the pH to the range specified.

5. The tablet of claim 1, wherein the sweetener is selected from at least one of the group consisting of polysaccharides, neotame, Sucralose, Acefulame K, Saccharin(s), Cyclomates, Apartame, Licorice root, Glycyrrhizin extract, Citrguar gum, guar gum, lactose, glyhrician, artificial polyols, natural polyols coming from fruits and trees, highly refined fruits, purified herbs, green tea, white tea, stevia, carrageenan, cranberry, natural pectin, alginic acid, xanthan gum, hyaluronic acid, chondroitin sulfate, gum Arabic, gum karaya, gum tradgacanth, chitosan, starch, dextrins, guar gum, manitol, sorbitol, isomalt and xylitol and mixtures thereof.

6. The tablet of claim 5, wherein the amount of sweetener ranges from about 0.01% to about 10% by weight, based on the weight of the tablet.

7. The tablet of claim 1, wherein the binder ranges from about 10% to about 90% by weight, based on the weight of the tablet.

8. The tablet of claim 1, wherein the binder is selected from the group consisting of polyols including manitol, sorbitol, isomal, xylitol and inverted sugars, calcium phosphate, calcium carbonate, sodium phosphate, sodium bicarbonate, sodium carbonate, sodium phosphate, potassium phosphate, potassium carbonate, polyethylene glycol and esters, and mixtures thereof.

9. The tablet of claim 1, wherein the amount of surfactant ranges from about 1.0% to about 8% by weight, based on the weight of the tablet.

10. The tablet of claim 1, wherein the amount of anthiocyanin extract ranges from about 0.1% to about 10% by weight, based on the weight of the tablet.

11. The tablet of claim 1, wherein the amount of abrasive ranges from about 0.1% to about 15% by weight, based on the weight of the tablet.

12. The tablet of claim 11, wherein the abrasive is selected from dehydrated silica, hydrated silica, calcium carbonate and surface treated carbonate salts and mixtures thereof.

13. A toothpaste tablet for use in brushing the user's teeth, comprising:
a binder in an amount ranging from about 10% to about 90% by weight, based on the weight of the tablet., a surfactant in amount ranging from about 1% to 5% by weight, based on the weight of the tablet, a cranberry extract in an amount ranging from 0.1% to about 10% by weight, based on the weight of the tablet, a silica abrasive in an amount ranging from about 0.1% to about 15% by weight, based on the weight of the tablet, and an artificial sweetener in an amount ranging from about 0.1% to about 10% by weight, based on the weight of the tablet.

14. The tablet of claim 13, which further includes a dental fluoride in an amount ranging from 0.01% to 5% by weight.

15. The tablet of claim 13, which further contains a buffer in an amount sufficient to adjust the pH when dissolved in the mouth to from about 6.0 to about 8.0.

16. The tablet of claim 15, wherein the cranberry extract provides an initial decrease in pH to below 6.0 and the buffering agent in an amount sufficient to restore the pH to the range specified.

17. A method of making a toothpaste tablet for use in brushing the user's teeth, comprising the steps of:
forming an admixture of a tablet binder in an amount sufficient to form a tablet, a surfactant in amount sufficient to reduce surface tension and increase the rate of dissolution of the tablet in contact with saliva, an anthiocyanad extract in an amount sufficient to reduce micro-adhesion of bacteria, an abrasive in an amount sufficient to mechanically remove debris from a tooth surface and debride plaque, and an artificial sweetener in an amount sufficient to adjust the flavor of the tablet when chewed by a user; and
compressing the admixture at a temperature of no more that about 78° F. to form a tablet.

18. The method of claim 17, which further includes the step of adding a buffer to the admixture in an amount sufficient to adjust the pH when dissolved in the mouth to from about 6.0 to about 8.0.

19. The method of claim 17, which further includes the step of adding a dental fluoride in an amount ranging from 0.01% to 5% by weight.

20. The method of claim 17, which further includes the step of adding a sweetener to the admixture selected from at least one of the group consisting of polysaccharides, artificial polyols, natural polyols, highly refined fruits, purified herbs, green tea, monogloicides, white tea, stevia, carrageenan, cranberry, natural pectin, alginic acid, xanthan gum, hyaluronic acid, chondroitin sulfate, gum Arabic, gum karaya, gum tradgacanth, chitosan, starch, dextrins, neotame, sucralose, acefulame K, saccharin, cyclomates, aspartame, licorice root, guar gum, manitol, sorbitol, isomalt, xylitol and mixtures thereof.

21. (canceled)

22. (canceled)

23. (canceled)

24. (canceled)
